(19) United States
(12) Patent Application Publication
(10) Pub. No.: US 2004/0087914 A1
(43) Pub. Date: May 6, 2004

(54) Spinal needle system

(76) Inventors: Vincent E. Bryan, Mercer Island, WA (US); Alex Kunzler, Issaquah, WA (US); Daniel R. Baker, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/616,864

(22) Filed: Jul. 9, 2003

Related U.S. Application Data
(63) Continuation-in-part of application No. 10/039,240, filed on Jan. 4, 2002.

(51) Int. Cl. A61M 25/00

(52) U.S. Cl. 604/264; 606/223; 600/217

(57) ABSTRACT

A system for grasping, holding, stabilizing, and selectively releasing tissue with minimal damage to the tissue. The system can further include a device that signals entry into the epidural space. A tubular member is provided having at a distal tip an annular surface surrounding a terminal port and at least one barb projecting at an angle from the annular surface for grasping and controlling the tissue. Each barb is formed having a sharp edge configured to grasp the tissue as the tubular member is rotated about its longitudinal axis. One implementation of the system includes a plurality of unidirectional barbs spaced around the annular surface. The system can further include an indicator mechanism that gives a visual and a tactile indication of when the tubular member, such as a cannula, encounters and penetrates tissue. The system facilitates the appropriate placement of an epidural or subdural catheter or patch of any kind.

### TECHNICAL FIELD

The present invention relates to spinal needles, and in particular, to an epidural cannula individually and in combination with an epidural stylet that stabilizes tissue during penetration by the stylet and provides visual and tactile indications of contact with and penetration of tissue.

### BACKGROUND OF THE INVENTION

Epidural cannula and spinal needles have been used for a variety of medical purposes, including extraction of cerebrospinal fluid (CSF) for laboratory tests and measurements, introduction of contrast or radionucleotide agents for diagnostic radiological testing, introduction of pharmaceutical agents into the subarachnoid space for therapeutic or anesthetic purposes, and facilitation of catheter placement within the subarachnoid and epidural spaces. While useful, spinal needles require extreme care to ensure their proper placement relative to the spinal dura mater and subarachnoid and epidural spaces.

Known spinal and epidural needles and the techniques of their placement, whether free hand or monitored radiologically, are associated with an unacceptably high incidence of complications. Improper placement results in such commonly encountered complications as post lumbar puncture spinal headaches, introduction of contrast agents into the subdural space rather than into the subarachnoid space, misplacement of an epidural catheter into the subarachnoid space, and epidural vessel hemorrhage, which may contaminate CSF samples. Such complications may interfere with the completion of reliable testing of CSF samples and proceeding with diagnostic tests. Misplacement of catheters relative to the subarachnoid and epidural spaces may also complicate the interpretation of diagnostic tests. For example, misplacement of the catheter can result in the introduction of contrast or radionucleotide agents into unintended spaces, such as the injection of a contrast agent into the subdural rather than the subarachnoid space during myelography. Misplacement of the catheter can also result in the administration of ineffective, toxic, or lethal dosages of anesthetic, antibiotic, chemotherapeutic, or other pharmaceutical or diagnostic agents. Furthermore, post lumbar puncture spinal headaches cause patients to suffer protracted periods of painful disability.

### SUMMARY OF THE INVENTION

The embodiments of the invention are directed to a device used in surgical, medical, and diagnostic procedures, treatments, and techniques, preferably by one trained in medical science and surgical arts, for grasping and holding tissue and releasing of the same with minimal tissue damage. In one embodiment, a spinal needle delivery system having a device for grasping tissue is provided. It is to be understood that the term “spinal needle” can encompass structural elements with or without a hollow longitudinal bore, and can include a tube, a cannula, a needle, a catheter, a stylet, and having either a blunt or sharp distal end. The system includes a tubular member, such as a cannula, having at a distal tip an annular surface surrounding a terminal port, and at least one barb projecting at an angle from the annular surface of the tubular member; each of the at least one barbs having a sharp edge configured to grasp the tissue as the tubular member is at least partially rotated about its longitudinal axis. Ideally, a plurality of unidirectional barbs are spaced around the annular surface of the tubular member.

In accordance with another aspect of the invention, an assembly is provided for tensioning the needle with respect to the cannula and for signaling in a visual and tactile manner the position of the needle. The assembly includes a spring-like tensioning member mounted in a housing that is attached to a proximal end of the cannula and configured to permit limited movement of a proximal end of the needle therein.

According to another embodiment of the invention, a method of using a spinal needle delivery system having a cannula with at least one barb projecting from a distal surface thereof is provided. The method includes: inserting the cannula through a first layer of tissue; detecting contact of the distal surface of the cannula with a second layer of tissue; and rotating the cannula in a first direction about its longitudinal axis to urge the at least one barb into engagement with the second layer of tissue.

In accordance with another aspect of the present invention, A surgical device is provided that includes a cannula having at a distal tip a terminal port and a mechanism for grasping and releasing epidural tissue without puncturing through the tissue.

In accordance with yet a further aspect of the present invention, a delivery system is provided that includes a tube having a longitudinal axial bore and, at a distal tip, an annular surface surrounding a terminal port, and member for grasping and releasing tissue, preferably without puncturing through the tissue; a housing secured to a proximal end of the tube, the housing having an internal cavity with an aperture formed in a proximal surface thereof opposite the proximal end of the tube; a tool sized and shaped to be slidably received within the bore of the tube and having a blunt distal tip portion sized to pass through the terminal port in the distal tip of the tube and a proximal end portion sized to pass through the aperture in the proximal surface of the housing, the tool mounted in the housing to move between an extended position wherein the distal tip portion extends beyond the distal tip of the tube and a retracted position wherein the distal tip portion is withdrawn inside the tube; and a resilient compression member mounted in the housing and configured to engage the tool when the tool is at an intermediate position between the extended position and the retracted position to thereby urge the tool into the extended position.

In accordance with still yet another aspect of the present invention, a method is provided that includes the steps of initially slidably receiving within a bore of the cannula a tool sized and shaped to be slidably received within the bore of the cannula and having a distal tip portion sized and shaped to pass through an annular port in the distal surface of the cannula; and after the at least one barb is engaged with the second layer of tissue, passing the distal tip portion of the tool through the annular port in the distal surface of the cannula.

In accordance with another aspect of the foregoing embodiment of the invention, a method of using a spinal needle delivery system that has a cannula having at least one barb projecting from a distal surface thereof and a blunt stylet projecting from a portal in the distal surface thereof under pressure from a resilient biasing member, the blunt stylet movable relative to the distal surface of the cannula by compression and expansion of the resilient biasing member; an indicator portion generating an indication as a function of a degree of projection of the blunt stylet relative to the portal in the distal surface of the cannula; and a cannula lock coupling an adhesive band to the cannula is provided. The method includes, in a previously perforated first layer of relatively high resistance tissue, enlarging the perforation sufficiently to permit entry of a distal tip of the blunt stylet; stabilizing the spinal needle delivery system relative to the enlarged perforation; advancing the distal tips of the blunt stylet and the cannula into and through the enlarged perforation in the layer of relatively high resistance tissue; using the indicator, determining that the distal tip of the blunt stylet has passed through the enlarged perforation in the layer of relatively high resistance tissue into a space of relatively low resistance; securing the cannula lock to the shaft of the cannula, thereby fixing the adhesive band relative to the cannula and advancing the distal tips of the blunt stylet and the cannula through the space of relatively low resistance and into contact with a second relatively high resistance tissue; using the indicator, determining that the distal tip of the cannula has contacted the second relatively high resistance tissue; rotating the cannula into an engaged position by rotating the cannula in a direction to engage the barbs with the second relatively high resistance tissue until resistance to continued rotation is encountered; supporting the cannula in the engaged position while advancing the cannula lock and adhesive band along the shaft of the cannula until the adhesive band contacts but does not depress the first layer of relatively high resistance tissue adjacent to the enlarged perforation; adhering the adhesive band to the first layer of relatively high resistance tissue; and supporting the cannula of the spinal needle delivery system.

### BRIEF DESCRIPTION OF THE DRAWINGS

FIG. 1 illustrates one embodiment of the invention implemented as a spinal needle delivery system.

FIG. 2A is an end view and FIG. 2B is a partial cross-sectional view of the distal end of the cannula illustrating one embodiment of the grasping barbs of the invention that project from the blunt annular surface of the cannula.

FIG. 3 illustrates an embodiment of the spinal needle delivery system of the invention that shows the blunt epidural space stylet installed in the blunt cannula.

FIGS. 4A and 4B are partial cross-sectional illustrations of the operation of a depth-limiting mechanism of the invention.

FIG. 5 is a cross-sectional illustration of the distal end of a spinal needle in cooperation with one embodiment of the blunt-tipped cannula of the invention.

FIG. 6 is a cross-sectional view and FIG. 7 is an end view illustrating the use of an epidural catheter guide of the invention in combination with the blunt-tipped cannula in accordance with one embodiment of the invention.

FIG. 8 illustrates a stopper stylet from in accordance with another useful aspect of the invention.

FIG. 9 illustrates the use of the illustrated embodiment of the spinal needle delivery system of the invention.

FIGS. 10A-10B illustrate in cross-section another embodiment of the spinal needle delivery system formed in accordance with the present invention.

FIGS. 11A-11B illustrate in an isometric view and partial side view, respectively, an alternative embodiment of the cannula tip formed in accordance with the present invention.

FIG. 12 illustrates in cross section another embodiment of the spinal needle delivery system formed in accordance with the present invention

FIGS. 13A-13B illustrate in cross-section and side views, respectively, a stylet stopper deliver system formed in accordance with another embodiment of the invention.

FIG. 14 illustrates an epidural catheter guide delivery system formed in accordance with another embodiment of the invention.

FIG. 15 illustrates a method of affixing the cannula to the patient in accordance with another embodiment of the invention.

FIG. 16 illustrates another method of affixing the cannula to the patient.

FIGS. 17A-B, 18-21, 22A-C, 23, 24A-B, 25, 26, 27A-C, and 28A-B are cross-sectional side views of additional embodiments of the invention.

### DETAILED DESCRIPTION OF THE INVENTION

The disclosed embodiments of the present invention are directed to a spinal needle delivery system that includes a device for grasping tissue composed of various materials. Although described herein as a tubular cannula for use in delivering a stylet to an epidural space in the human body, the tissue-grasping device is not intended to be limited to use in connection with dura mater tissue, or with bodily tissue generally. Rather, the tissue-grasping device of the invention is generally applicable to stabilize a delivery system for delivering a tool through various membranes and tissues.

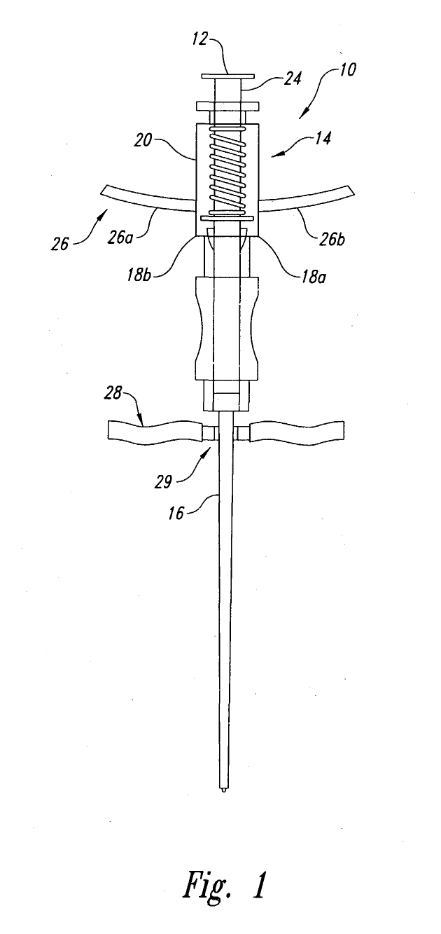

FIG. 1 illustrates one embodiment of the invention implemented as a spinal needle delivery system 10, that overcomes the complications experienced with prior spinal needles. The system 10 both identifies contact with and stabilizes the dura mater tissue. Tissue contact is identified by a blunt-tipped epidural space stylet (or simply stylet) 12 advanced through a cannula under the influence of a biasing mechanism 14. The system 10 signals the stylet's entrance into the epidural space and its contact with the dura mater by activating a combination tactile and visual signal portion of the stylet 12. The system 10 also stabilizes the dura mater by attachment of the distal tip of a cannula 16 to the dura mater tissue. The identification of and attachment to the dura mater tissue provides directional control and depth control for a spinal needle or catheter passed through the tubular cannula 16 and into the subarachnoid space. This in turn facilitates the appropriate placement of a catheter or blood patch into the epidural or subdural space.

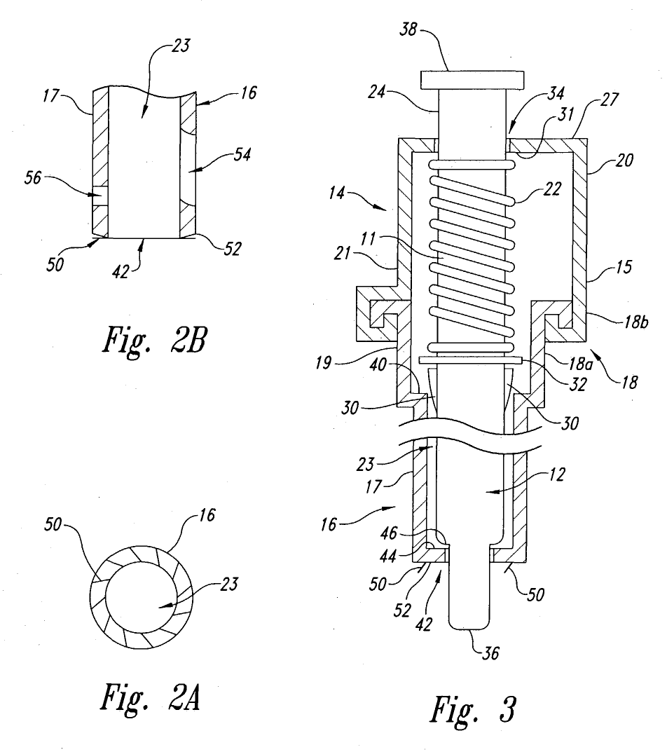

As shown in FIGS. 2A-2B, the cannula 16 is a blunt-tipped tubular body 17 having a longitudinal axis and terminating in a distal annular port 42 surrounded by sharpened hooks or barbs 50. These barbs 50 may also comprise sliver or scale-like serrations configured to at least partially penetrate the tissue. The blunt distal tip 52 of the cannula 16 prevents penetration of the membrane or tissue. The barbs 50 are configured for grasping and stabilizing tissue encountered at the distal tip 52 of the cannula 16. The barbs 50 are fashioned to engage a membrane or layer of tissue when the cannula 16 is partially rotated about its longitudinal axis, preferably in a clockwise direction relative to the tissue, and to release or disengage from the tissue when the cannula 16 is rotated in a reverse direction, preferably in a counterclockwise direction.

FIGS. 2A and 2B together illustrate one embodiment of the grasping barbs 50 of the invention that project from the blunt distal tip 52 of the cannula 16. According to the embodiment illustrated, the barbs 50 are configured as unidirectional sharpened sliver or scale-like serrations distributed around annular surface of the distal tip 52 surrounding the terminal port 42 and are circumferentially aligned relative to the longitudinal axis of the bore 23 of the cannula 16.

As is more clearly illustrated in FIG. 2B, the barbs 50 project from the blunt tip 52 of the cannula 16 at a shallow angle in order to more effectively grasp tissue. The barbs 50 are alternatively fashioned in any suitable form for grasping tissue as the cannula 16 is partially rotated about its longitudinal axis. For example, the barbs 50 are configured to insert into and engage the outer portion of the dura mater when the cannula 16 is partially rotated in the clockwise direction. According to one embodiment of the invention, rotation of the cannula 16 by about 30 degrees fully engages the barbs 50 in the dura mater tissue. While the barbs 50 are configured to grasp the tissue, a further consideration is that the barbs 50 release the dura mater tissue when rotated in the opposite or counterclockwise direction. Furthermore, the barbs 50 are configured such that the dura mater tissue is not perforated during either engagement or release. The blunt tip 52 of the cannula 16 further facilitates the ability of the barbs 50 to grasp tissue without perforating or puncturing.

The annular port 42 of the cannula 16 is sized to pass a spinal needle or a catheter therethrough and into the subarachnoid space. Various other lateral ports may be provided for the passage of an epidural catheter or a blood patch into the epidural space, as described in detail below.

As shown in FIG. 3, an interior portion of the distal tip 52 of the cannula 16 optionally includes a peripheral ring 44 useful as a depth-limiting mechanism for a spinal needle of another tool. For example, the peripheral ring 44 may also engage a stopper stylet or an epidural catheter guide of the invention; both described in detail below.

In one embodiment, the cannula 16 may be about three and one-half inches in length. A proximal end 19 of the cannula 16 is structured for attachment of a distal portion 15 of the biasing mechanism 14. For example, the proximal end 19 of the cannula 16 is fitted with one portion 18a of a locking mechanism or connector 18. The biasing mechanism 14 includes, for example, a housing 20 containing a biasing member 22 implemented as a resilient compression member. The housing 20 and biasing member 22 form a sleeve that is fitted around a proximal portion 11 of the stylet 12. A distal end 21 of the housing 20 is fitted with a mating portion 18b of the connector 18. The mating portions 18a and 18b of the connector 18 are structured to be releaseably coupled when the stylet 12 is slidably received within the cannula 16.

The blunt-tipped epidural space stylet 12 of the invention is sized and shaped to be slidably received into a longitudinal axial bore 23 of the cannula 16. The stylet 12 is structured with a blunt or rounded distal tip 36 that, when inserted into the cannula 16, passes through the distal annular port 42 and projects beyond the distal tip 52 of the cannula 16. The biasing mechanism 14 urges the distal tip 36 of the stylet 12 to normally extend or project from the distal tip 52 of the cannula 16. The near or proximal end 11 of the stylet 12 is coupled to the biasing mechanism 14 but is free to move within the bore 23 of the cannula 16, within predetermined limits. An indicator portion 24 at the proximal end 11 of the stylet 12 is free to move in and out of an aperture 34 at the proximal end 27 of the housing 20.

In this embodiment, the housing 20 includes a finger rest 26 implemented, for example, as a pair of lateral wing extensions 26a and 26b, which are useful for supporting and operating the spinal needle delivery system 10. The lateral wing extensions 26a and 26b are sized for grasping by the fingers and to facilitate insertion of the cannula 16 into tissue.

An adhesive band 28 may be attached to the shaft on the exterior of the cannula 16 by a cannula lock 29. The adhesive band 28 is to stabilize the cannula 16 relative to the patient's body and prevent counter-rotation thereof.

In operation, the distal tip 52 of the cannula 16 is inserted through a previously incised perforation in the skin and muscle tissue until the distal tip 52 of the cannula 16 contacts the dura mater surrounding the subarachnoid space. As the cannula 16 and stylet 12 are advanced, the skin and underlying muscle tissue present a relatively high resistance that causes the stylet 12 to compress or “load” the resilient biasing mechanism 14. Loading the biasing mechanism 14 causes the indicator portion 24 at the proximal end 11 of the stylet 12 to project from the proximal end 27 of the housing 20. The indicator portion 24 thus presents a tactile and visual indication that the distal tip 52 of the cannula 16 and stylet 12 are advancing through relatively high resistance muscle tissue.

When a lesser resistance is encountered, such as the epidural space between muscle tissue and the dura mater, the biasing mechanism 14 automatically advances or “discharges” the distal tip 36 of the stylet 12 for a limited distance beyond the distal tip 52 of the cannula 16. Thus, upon entering the epidural space, the resilient biasing mechanism 14 is “unloaded,” which permits the indicator portion 24 at the proximal end 11 of the stylet 12 to retract into the proximal end 27 of the housing 20. Retraction of the indicator portion 24 indicates entry of the distal tip 36 of the stylet 12 into the epidural space.

The cannula 16 is then advanced over the stylet 12 until the distal tip 52 of the cannula 16 encounters the dura mater, whereupon the cannula 16 is rotated about its longitudinal axis to engage the dura mater. This stabilizing the dura mater tissue so that a spinal needle, a catheter, or another tool can be delivered through the various membranes and tissues to the appropriate site. As the cannula 16 is advanced over the stylet 12 to contact the dura mater, the stylet 12 can be withdrawn, sometimes simultaneously.

The optional adhesive band 28, if present, is adhered to the outer membrane through which the distal tip 52 of the cannula 16 is inserted. For example, in a spinal needle application, the adhesive band 28 is adhered to the patient's skin to help stabilize the installed cannula 16. After insertion and engagement of the cannula 16, the adhesive band 28 is advanced to a position along the cannula 16 near to the skin perforation at the entry point of the cannula 16. The adhesive band 28 is then adhered to both the cannula 16 and the patient's skin, thereby helping to maintain the depth and orientation of the cannula 16 relative to the perforation.

Loosening the adhesive band 28 and rotating the cannula 16 in the reverse direction until the barbs 50 are released from the tissue disengages the cannula 16. The cannula 16 is retrieved by withdrawal from the incised perforation.

FIG. 3 illustrates the blunt epidural space stylet 12 installed in the cannula 16. The biasing mechanism 14 is implemented using the resilient compression member 22 captured within the housing 20. The resilient compression member 22 is, for example, implemented as a spring or spring-like mechanism, or other resilient material sized and shaped to provide a resistive force.

According to this embodiment of the invention, the spring 22 is positioned between one or more engagement blocks 30 on the stylet 12 and an inner surface 31 at the proximal end 27 of the housing 20. The engagement blocks 30 are optionally implemented as one or more rigid lateral protrusions 30 that enlarge the outside diameter of the stylet 12. The engagement blocks 30 are sized to fit within the tubular bore of the cannula 16. The engagement blocks 30 are located on the stylet 12 at a position that will interact with the spring 22, either directly or, in this case, through a reaction member 32. The optional reaction member 32 is, for example, an annular disc having an inner diameter sized to slidably engage the outer diameter of the stylet 12, but to interfere with the engagement blocks 30.

In this embodiment of the invention, the housing 20 of the biasing mechanism 14 is implemented as mating male and female portions of a modified luerlock connector. A female portion of the luerlock connector is provided as the connector portion 18a attached to the proximal end of the cannula 16. The distal end of the housing 20 is formed of the male connector portion 18b of the luerlock connector. The female 18a and male 18b connector portions of the modified luerlock connector interconnect to form the locking mechanism 18.

The spring 22 is initially compressed between the reaction member 32 on the body of the stylet 12 and the inner proximal surface of the housing 20 to provide a predetermined amount of pre-load force on the stylet 12. The normal expansion of the compressed spring 22 urges the distal tip 36 of the stylet 12 to project from the distal tip 52 of the cannula 16. The spring 22 is selected to store an amount of pre-load force appropriate to the particular application for which the invention is practiced. For example, when implemented for a total spinal needle application, the spring 22 is selected to have a spring force that is less than the force required to advance the distal tip 52 of the cannula 16 through the perforation in the skin and the underlying muscle tissue. Advancement through the skin and muscle tissue thus compresses or “loads” the spring 22 with an increased pre-load and causes the indicator portion 24 to project through the oversized aperture 34 in the proximal end 27 of the housing 20.

The spring 22 is further selected to have a spring force greater than the lower resistance within the epidural space. Because the dura mater is a pulsating tissue due to the pulsing of blood vessels in the spinal cord and brain, the epidural space is periodically subjected to negative pressure. This negative pressure will act to draw the stylet 12 into the epidural space. Upon encountering such negative pressure or a lower resistance tissue, the spring 22 unloads to its normally expanded configuration and discharges the distal tip 36 of the stylet 12. For example, the distal tip 36 of the stylet 12 is discharged from the terminal port 42 in the distal tip 52 of the cannula 16 by approximately three to four millimeters. The extended indicator portion 24 of the stylet 12 is retracted by a similar amount through the aperture 34 into the proximal end 27 of the housing 20.

The distance by which the distal tip 36 of the stylet 12 is moved by the spring 22 is also selectable to satisfy various applications. According to one embodiment of the invention, the housing 20 cooperates with the a lateral protrusion on the proximal end portion 11 of the stylet 12 to implement a “depth-limiting” mechanism that controls the distance by which the distal tip 36 of the stylet 12 is projected from the distal tip 52 of the cannula 16. For example, a proximal cap 38 on the proximal end portion 11 of the stylet 12 is sized with an outer diameter larger than the aperture 34 in the proximal end 27 of the housing 20. The aperture 34 thus restricts the motion of the stylet 12 toward the distal tip 52 of the cannula 16 by interfering with the proximal cap 38.

Alternatively, the engagement blocks 30 are sized larger than a peripheral annular seat 40 portion of an inner distal surface of the housing 20. The peripheral seat 40 interferes with the oversized engagement blocks 30, thus providing a depth-limiting mechanism for the distal tip 36 of the stylet 12 relative to the distal tip 52 of the cannula 16.

According to another embodiment of the invention, the engagement blocks 30 are sized larger than the inner diameter of the bore 23 of the cannula 16. The proximal opening into the cannula 16 interferes with the oversized engagement blocks 30. The proximal surface of the cannula 16 thus provides a depth-limiting mechanism for the distal tip 36 of the stylet 12 relative to the distal tip 52 of the cannula 16.

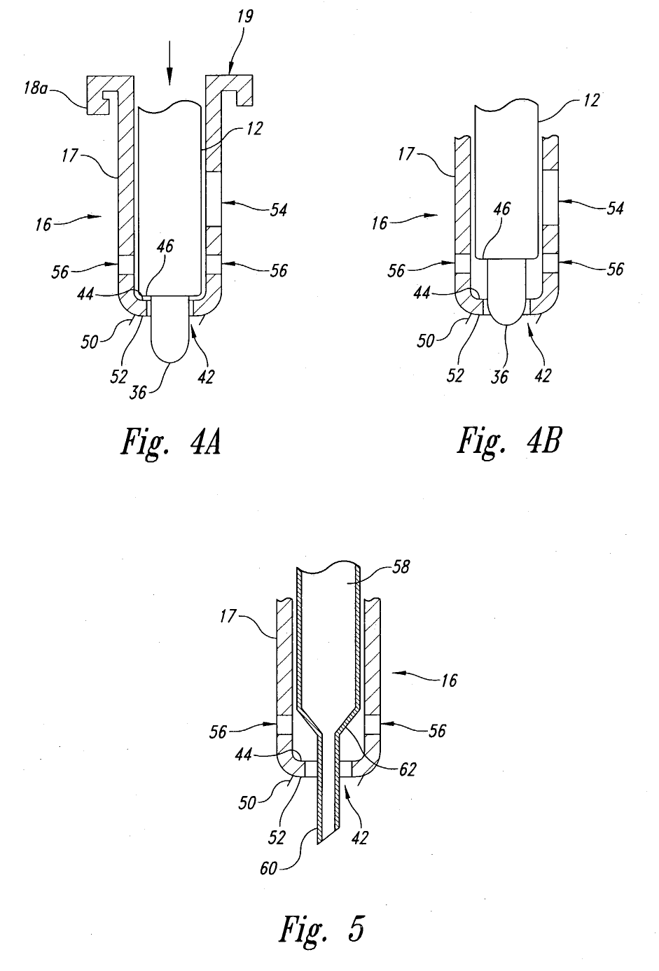

FIGS. 4A and 4B together illustrate another depth-limiting mechanism of the invention. In FIG. 4A, the epidural space stylet 12 of the invention is shown in a discharged state, wherein the spring force of the biasing mechanism 14, as indicated by the directional arrow, urges the blunt distal tip 36 of the stylet 12 to advance through the terminal port 42 in the cannula 16. The peripheral ring 44 is sized with an inner diameter somewhat smaller than the inner diameter of the tubular cannula 16. The peripheral ring 44 thus provides a depth-limiting mechanism for the distal tip 36 of the stylet 12 relative to the distal tip 52 of the cannula 16. For example, the stylet 12 is provided with a shoulder portion 46 at a predetermined setback distance from the extent of the blunt distal tip 36. While the blunt distal tip 36 is sized to pass through the reduced diameter of the terminal port 42, the shoulder portion 46 is sized to encounter the inner peripheral ring 44, which restricts projection of the blunt distal tip 36 to a predetermined distance beyond the distal tip 52 of the cannula 16.

FIG. 4B illustrates the epidural space stylet 12 in a loaded state, wherein a resistance encountered at the distal tip 52 of the cannula 16 is sufficient to overcome the spring force provided by the biasing mechanism 14. In such circumstance, the blunt distal tip 36 of the stylet 12 is pushed back inside of the cannula 16, lifting the shoulder 46 off of the inner peripheral ring 44 and storing a predetermined pre-load in the biasing mechanism 14 as a function of its spring rate.

As discussed above, the pre-load force is stored in the biasing mechanism 14 until the distal tip 52 of the cannula 16 passes through the high resistance tissue into a space, such as the epidural space, which presents a resistance that is less than the spring force of the biasing mechanism 14.

The distal tip 36 of the epidural space stylet 12 is sufficiently blunt to avoid inadvertently perforating tissue, such as the dura mater tissue, as the cannula 16 is advanced through the epidural space and into contact with the dura mater.

The distal or terminal port 42 of the cannula 16 is sized to permit the passage of a spinal needle or a catheter into the subarachnoid space. Various other ports are provided in the lateral surfaces of the distal tip 52 of the cannula 16. As shown in FIGS. 4A and 4B, a lateral epidural port 54 is provided adjacent to the distal tip 52 and is sized to pass an epidural catheter or blood patch. Optionally, one or more smaller auxiliary lateral ports 56 are provided near the distal tip 52 of the cannula 16. The smaller auxiliary ports 56 are useful, for example, for administering blood patches.

FIG. 5 illustrates an embodiment of the blunt-tipped cannula 16 of the invention, including the peripheral ring 44 within the interior of the terminal port 42, as described above. The peripheral ring 44 is sized with an inner diameter somewhat smaller than the inner diameter of the tubular cannula 16. The peripheral ring 44 thus provides a depth limiting mechanism for a tool acting at or through the distal tip 52 of the cannula 16. As shown in FIG. 5, the peripheral ring 44 is a depth limiting mechanism for a spinal needle 58. The spinal needle 58 or another tool intended to operate beyond the distal tip 52 of the cannula 16 includes an active portion 60 that is sized to pass through the reduced diameter terminal port 42. The maximum extension of the active portion 60 is limited to about 6 mm by a shoulder 62 that is sized to encounter the inner peripheral ring 44. Interference between the shoulder 62 and the inner peripheral ring 44 restricts further extension of the active portion 60.

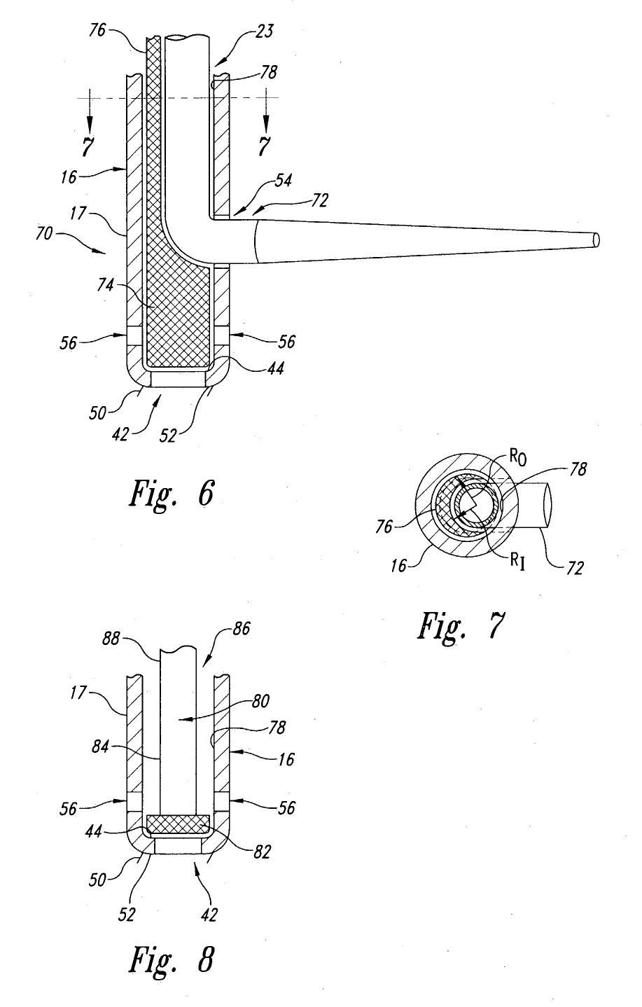

FIGS. 6 and 7 together illustrate the use of an epidural catheter guide 70 of the invention in combination with the blunt-tipped cannula 16 of the invention. The epidural catheter guide 70 is sized small enough to be slidingly received within the tubular bore 23 of the cannula 16, but sufficiently large to engage the inner peripheral ring 44 partially obstructing the terminal port 42, which effectively restricts further advancement of the epidural catheter guide 70. The epidural catheter guide 70 is configured to direct an epidural catheter 72 through the lateral epidural port 54. The epidural catheter guide 70 is configured, for example, with a plug portion 74 at the distal end of a shaft 76. The plug portion 74 is sized and shaped to interfere with the inner peripheral ring 44 of the terminal port 42 and limit the further advancement of the shaft 76.

The shaft 76 intersects with the plug portion 74 in a curved configuration that urges the catheter 72 into a directional change relative to the cannula 16. Furthermore, the plug portion 74 is sized to provide the directional change in proximity to the lateral epidural port 54 in the cannula 16.

As illustrated in FIG. 7, the epidural catheter guide 70 is configured to combine with the interior wall surface 78 of the cannula 16 to form a tube-like channel that slidably receives the tubular catheter 72 and directs it down to and through the lateral epidural port 54.

The shaft 76 of the epidural catheter guide 70 is, for example, formed to have a partial tubular shape with an outer radial dimension R₀ and sized to be slidably received within the tubular bore 23 of the cannula 16. The shaft 76 has an inner radial dimension R₁ sized to permit easy advancement of the epidural catheter 72 between the epidural catheter guide 70 and the inner wall surface 78 of the cannula 16. In operation, the inner radial surface of the shaft 76 coordinates with the inner wall surface 78 of the cannula 16 to direct the catheter 72 down to and through the lateral epidural port 54 and into the epidural space.

FIG. 8 illustrates another aspect of the blunt-tipped cannula 16 of the invention. A stopper stylet 80 of the invention is inserted into the previously stabilized cannula 16. According to one embodiment of the invention, the stopper stylet 80 includes a stopper 82 positioned at a distal tip of a shaft 84. The stopper 82, which is formed of rubber or another resilient material, is sized to be slidably received within the tubular bore of the cannula 16, while its advancement beyond the distal tip 52 of the cannula 16 is restricted by interference with the inner peripheral ring 44. Furthermore, the stopper 82 is sized small enough to avoid obstructing the one or more auxiliary lateral ports 56.

The shaft 84 of the stopper stylet 80 is concentric with the stopper 82. The outer surface 88 of the shaft 84 thus cooperates with the inner wall surface 78 of the cannula 16 to form an annular passage or channel 86 therebetween that communicates with the auxiliary lateral ports 56 through which blood or another fluid may flow.

### Operation

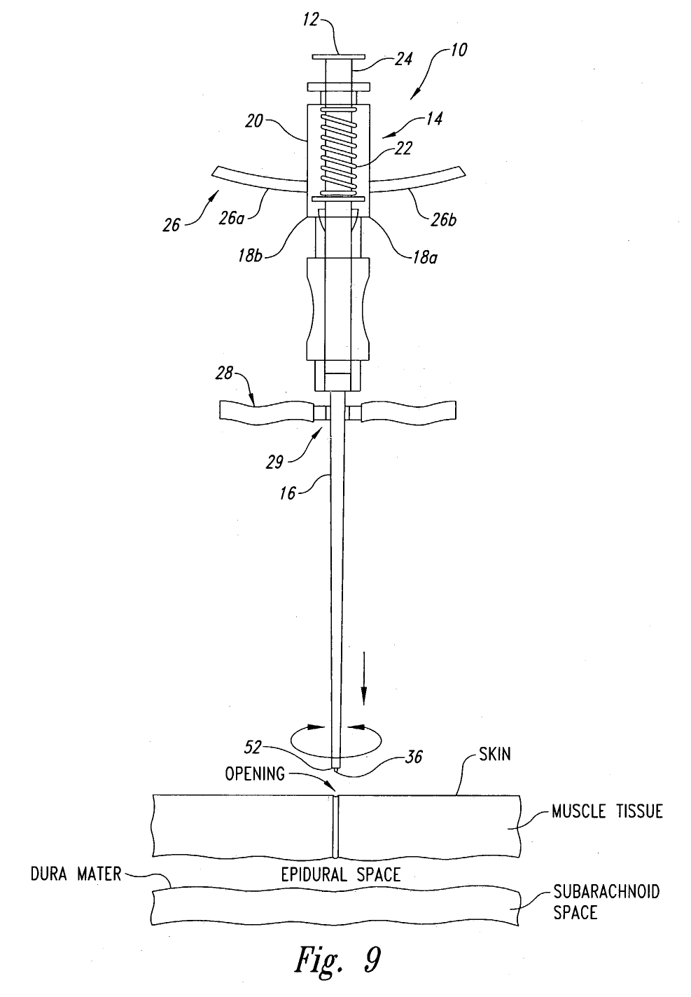

FIG. 9 illustrates the use of the described embodiment of the spinal needle delivery system 10 with a patient who is in any of the lateral decubitus, sitting, and prone positions. An appropriate antiseptic preparation is completed on the patient's skin. A local anesthetic is administered to anesthetize the tissues, inclusive of the lumbosacral fascia located just cephalad to the spinous process, in the midline, of the selected interspinous process space. The skin opening is enlarged to admit the distal tip 36 of the blunt stylet 12. The blunt cannula 16 containing the blunt stylet 12 is grasped by the pair of lateral wing extensions 26a and 26b using the thumb and index fingers of both hands. The middle, ring, and small fingers of both hands are extended and applied to the paravertebral skin surfaces bilaterally to provide a stabilizing scaffold for the cannula 16 and stylet 12 as they are gradually advanced through the enlarged skin opening.

Firm but steady pressure is applied to advance the needle delivery system 10 into and through the enlarged skin opening. For example, the needle delivery system 10 is advanced at the rate of approximately five millimeters per second for about the first four centimeters and more slowly thereafter, maintaining the needle delivery system 10 in the midline position at all times. As resistance to the advancement of the needle delivery system 10 is encountered, the distal tip 36 of the blunt stylet 12 is forced into the terminal port 42 in the distal tip 52 of the cannula 16, thereby compressing the spring 22 of the biasing mechanism 14. Simultaneously, the indicator portion 24 of the proximal end 11 of the stylet 12 is projected from the proximal end 27 of the housing 20. The spinal needle delivery system 10 thus provides a tactile and visual indication that the distal tip 52 of the cannula 16 is engaged in tissue that resists the advancement of the needle delivery system 10 with a greater force than the spring force of the biasing mechanism 14. In other words, projection of the indicator portion 24 from the housing 20 indicates that the distal tip 52 of the cannula 16 is advancing through skin and muscle tissue.

As the distal tip 52 of the cannula 16 enters the epidural space between the muscle tissue and the dura mater, the spring force of the biasing mechanism 14 overcomes the lower resistance, and the blunt distal tip 36 of the stylet 12 is urged outwards through the terminal port 42 of the cannula 16. Simultaneously, the indicator portion 24 at the proximal end 11 of the stylet 12 moves partially or completely into the aperture 34 in the proximal end 27 of the housing 20, thereby indicating that the epidural space as been penetrated. The lateral wing extensions 26a and 26b are released by the user. The cannula 16 is grasped and advanced along the shaft of the stylet 12 about three to four millimeters, while the cannula 16 is rotated in a direction to engage the barbs 50 with the dura mater tissue, for example, in a clockwise direction. The advancing and rotating of the cannula 16 is curtailed when resistance to continued rotation is encountered. The cannula 16 is supported in the engaged position while the cannula lock 29 and the skin adhesive band 28 are advanced along the shaft of the cannula 16 until the adhesive band 28 contacts but does not depress the skin. The cannula lock 29 is locked to the shaft of the cannula 16 to fix the adhesive band 28 relative to the cannula 16. The adhesive strips of the adhesive band 28 are adhered to the skin. Further support of the cannula 16 is unnecessary.

After the procedure is complete, the spinal needle delivery system 10 is removed in reverse order. The adhesive band 28 is separated from the skin, the barbs 50 are disengaged from the dura mater by reverse rotation of the cannula 16, and the cannula 16 is retrieved from the perforation.

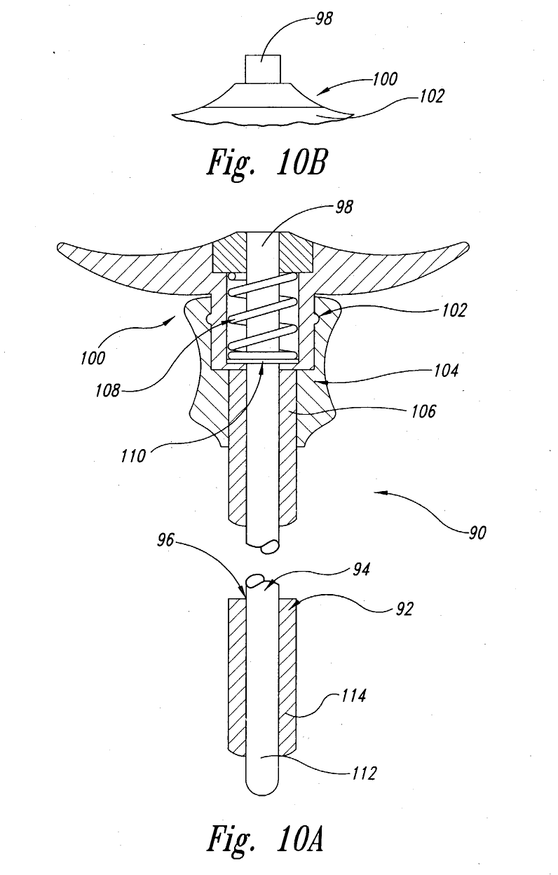

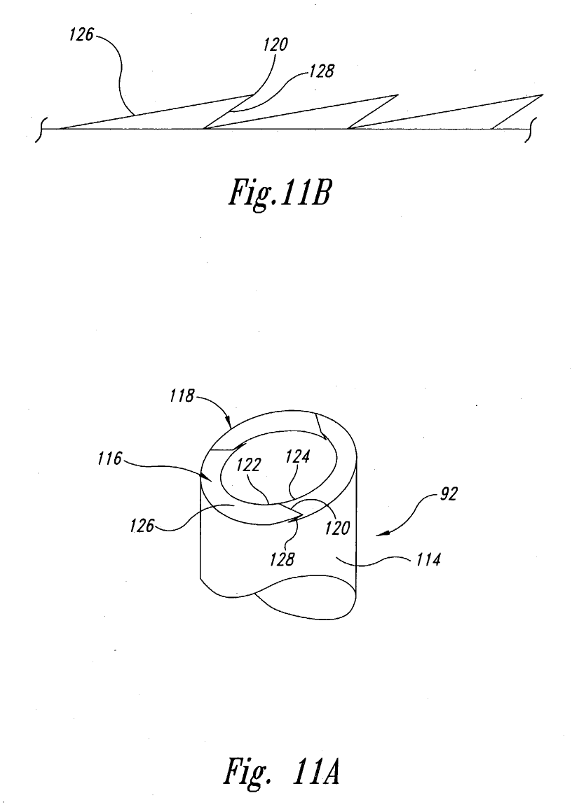

Turning next to FIGS. 10A-10B, shown therein is another embodiment of a spinal needle delivery device 90 that includes a cannula 92 having a blunt stylet 94 slidably mounted within a longitudinal axial bore 96. The stylet 94 has a proximal end 98 mounted within a housing 100. The housing 100 comprises a rotationally indexing male-type luer lock fitting 102 engaging a female-type luer lock coupling 104 in which a proximal end 106 of the cannula 92 is mounted. A coil spring 108 inside the male-type fitting 102 acts upon a disc 110 attached to the stylet 94 to urge the distal end 112 of the stylet 94 to project out of the distal end 114 of the cannula 92. When the stylet 94 encounters resistance that overcomes the force of the spring 108, the proximal end 98 of the stylet 94 projects out of the top of the housing 100, as shown in FIG. 10B.

FIGS. 11A-11B show an alternative configuration for the distal end 114 of the cannula 92. Here, the annular face 116 of the cannula 92 has three barbs 118 formed thereon. It is to be understood that additional or fewer barbs may be used, and their configuration may vary in order to accommodate certain tissues. The barbs 118 in this configuration each have first radially oriented side 120 and a second side 122 that converge to form a point 124. The first side 120 is formed by the intersection of the top side 126 and the bottom side 128 of the barb 118, as shown in FIG. 11A and in the side view of the barbs 118 in FIG. 11B, which shows a side view of the annular face 116 in rolled out configuration.

In FIG. 12, a spinal needle delivery device 130 is shown having a depth limited spinal needle assembly 132 mounted to the housing 100 using the male-type luer lock fitting 102 and corresponding female-type coupling 104 described above. The needle 134 projects out of the distal end 136 of the cannula 138 in which are formed a catheter port 140 and a pair of blood ports 142.

The blood ports 142 are used with the device 144 shown in FIG. 13, wherein a hollow stylet stopper 146 is slidably received within the cannula 138. The stopper 146 has a tip 148 with a circumscribing channel 150 formed therein. A transverse opening 152 formed through the channel intersects with a longitudinal axial bore 154 of the stylet stopper 146. A rubber membrane 156 covers the proximal end 158 of the stylet stopper 146 that is mounted in the male-type fitting 102.

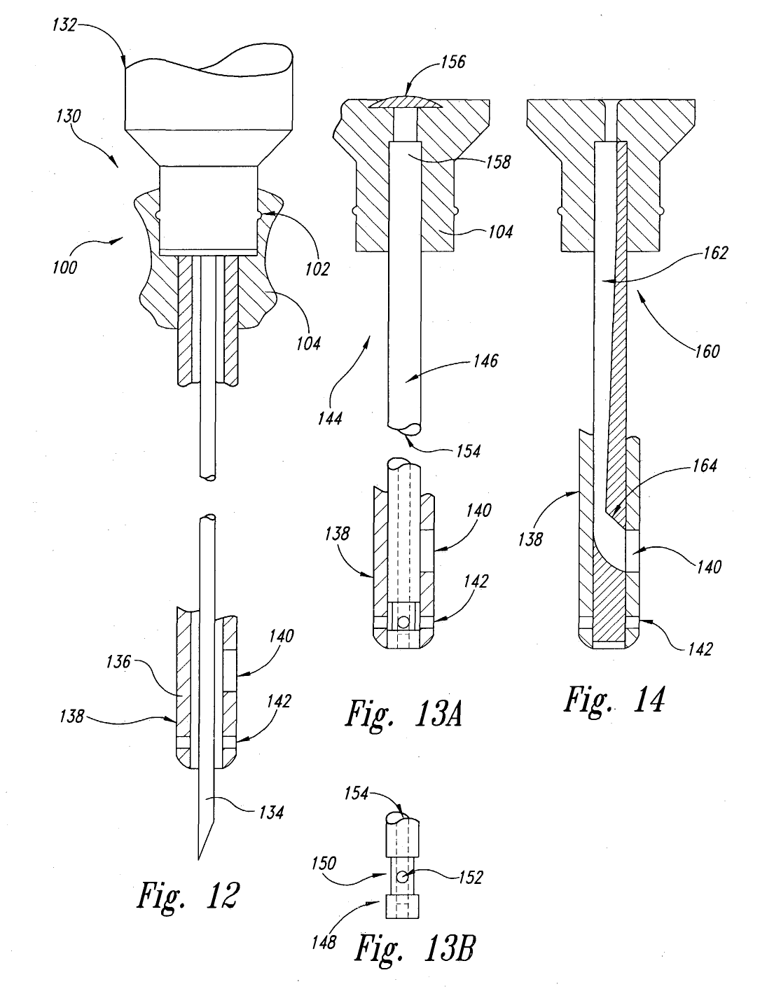

The catheter port 140 is used with the device 160 shown in FIG. 14. An epidural catheter guide 162 is used in conjunction with the male-type fitting 102 and the cannula 138 to intersect with the catheter port 140 for the passage of fluids. A sidewall 164 of the catheter guide intersects the catheter port 140 at about a 45-degree angle to enhance the flow of fluids.

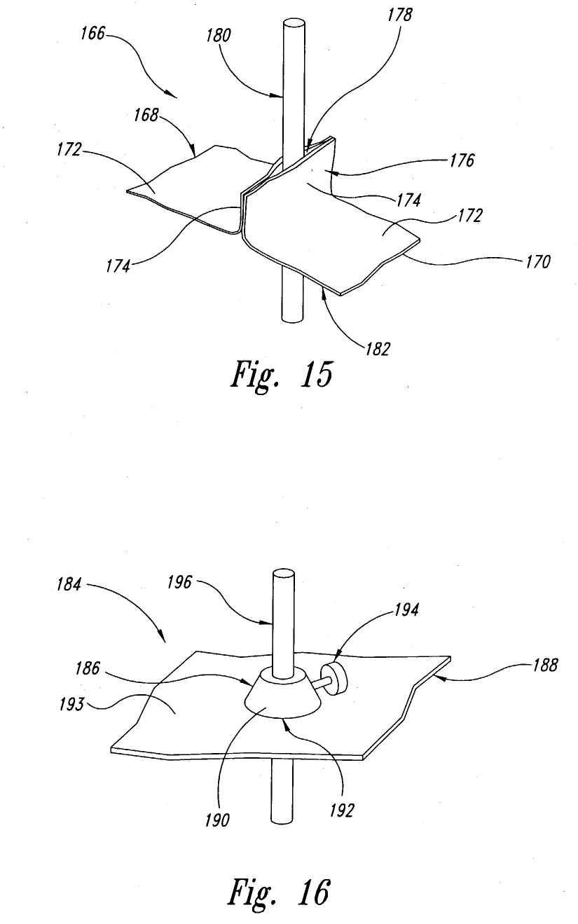

FIG. 15 shows an alternative method of affixing the spinal needle delivery device to the patient's skin. An attachment system 166 comprises first and second adhesive pads 168, 170, each formed in an L-shape to have a first segment 172 sized for attachment to the patient's skin (not shown) and a second segment 174 folded upward for attachment to the mating second segment 174, such as by bonding stitches 176 or other conventional fastening method. In this manner, an opening 178 is formed between the second segments in which the cannula 180 is inserted. Pressure sensitive adhesive on the second segments 174 affixes the cannula 180 to the two pads 168,170. An adhesive is used on the underside 182 of the pads 168,170 for attachment to the patient's skin.

Alternatively, another attachment system 184 is shown in FIG. 16 in which a locking block 186 is mounted to an adhesive skin patch 188. More particularly, the locking block comprises a truncated cone base 190 having a bottom surface 192 affixed to the top surface 193 of the skin patch, such as by adhesive. A threaded fastener 194 is threadably received in the base 190 to bear against and hold the cannula 196 in place. An adhesive on the bottom surface of the patch 188 holds the patch to the patient's skin (not shown).

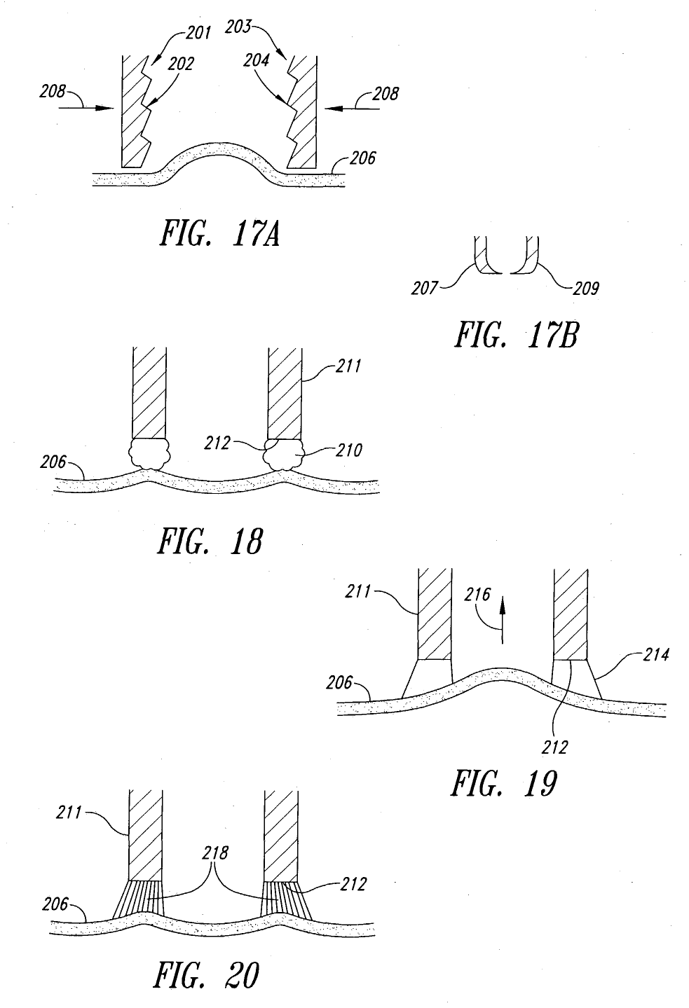

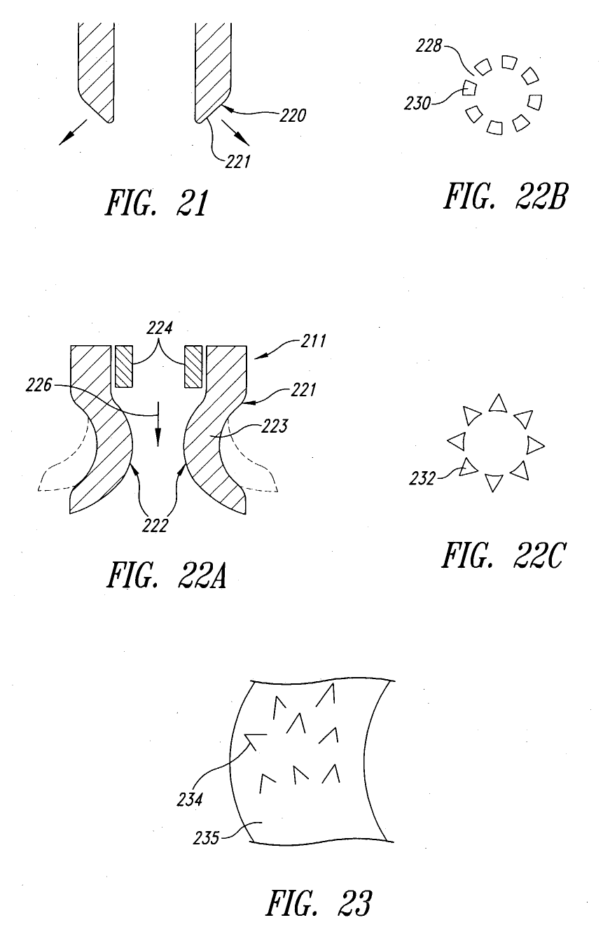

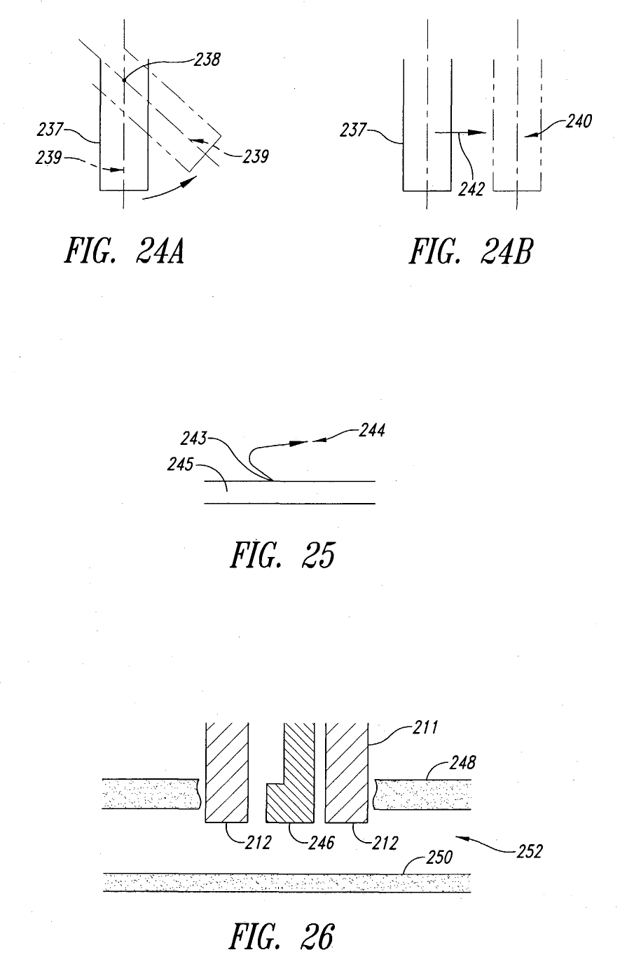

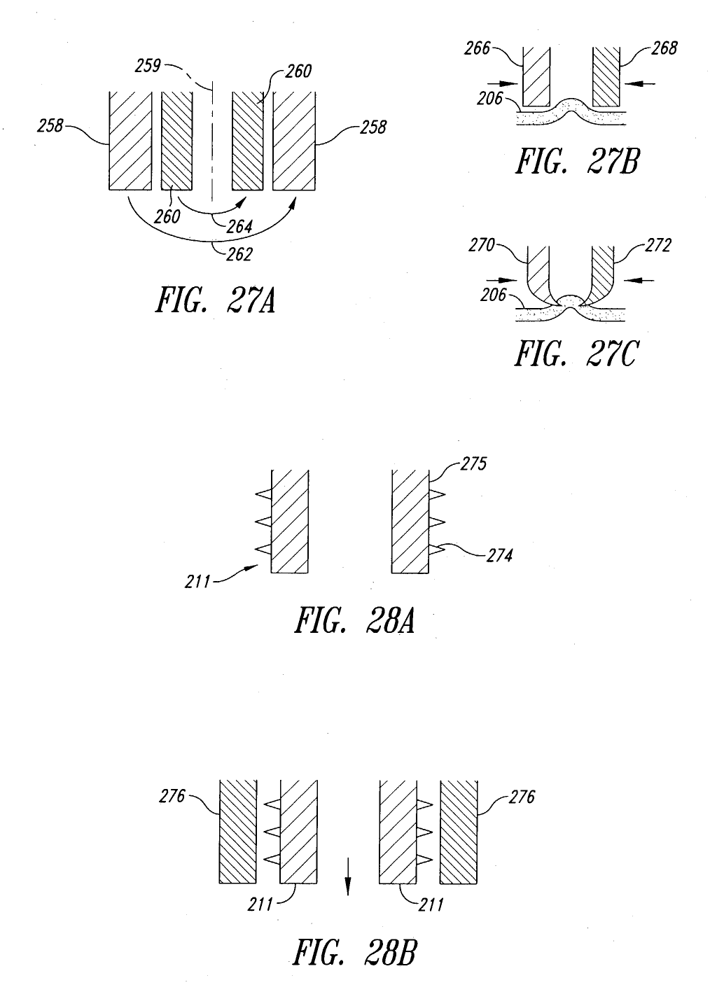

An alternate method for grasping tissue, shown in the embodiment of FIG. 17A, is where two surfaces 201, 203 come together (as indicated by the arrows 208 in the figure and retain some tissue 206 between them through application of a closing force). The embodiment shown in FIG. 17A includes serration-like machined surfaces 202, 204 that increase the holding ability of the surfaces but are not required to function. While the shape of the surfaces 201, 203 is shown to be flat, it is to be understood that other shapes may be used.

Instead of just two surfaces coming together, as in the embodiment shown in FIG. 17A, FIG. 17B shows a barb-like appendages on each surface 207 and 209. This is a more aggressive method of capturing the tissue for grasping and control. The size, shape, and number of barb-like appendages would be dependent on the type of tissue, what is to be done with the tissue, and the location of the tissue, among other variables.

FIG. 18 shows another embodiment for grasping tissue that uses an adhesive 210 that is placed on the surface that is to adhere to the tissue 206, in this case the distal face 212 at the distal end of the needle 211. Use of other delivery systems, such as an adhesive pad attached to the distal face 212 of the needle 211 could be incorporated rather than delivering the adhesive 210 to the distal face 212 of the needle 211. Currently, the amount of adhesive would have to be closely controlled so as to limit damage upon removal. As methods for controlling adhesion become more sophisticated in the ability to adhere and release, then that technology could be incorporated into this embodiment.

In yet another embodiment for grasping tissue, a pressure differential can be used. In FIG. 19 a flexible skirt 214 is shown attached to the distal face 212 of the needle 211. A mechanism for reducing the pressure inside of the needle 211 is applied to the proximal end (not shown) of the needle 211, resulting in the tissue 206 moving towards the proximal end, i.e., in the direction shown by the arrow 216. The flexible skirt 214 remains solidly in contact with the tissue 206 while the pressure differential exists; and when that differential is removed, then the two can separate easily. The skirt 214 is shown here, but may not be necessary depending on the strength of the tissue 206, size of the needle 211, and other design factors.

In the embodiment shown in FIG. 20, gecko feet 218 utilizing van der Waals forces are employed for adhering to walls. This mechanism can be reversed quickly (i.e., the feet 218 can disengage easily and swiftly) and is a function of the specific geometry of the feet 218 rather than surface chemistry. Applying a material with similar characteristics to the distal face 212 of the needle 211 provides good adhesion and enables controlled disengagement when needed to grasp tissue. [e.g., see: Autumn, Sitti, et al., “Evidence for van der Waals adhesion in gecko setae,” Proc. Nat. Acad. Science, 99(19): 12252-12256, 12 Sept. 2002, www.pnas.org/cgi/doi/10.1073/pnas.192252799.]

In the embodiment described in conjunction with FIG. 17B, barb-like appendages were placed on the perpendicular surface on the distal end of the needle to grasp tissue; however, in an alternative embodiment shown in FIG. 21, an external chamfer 221 is machined onto the distal end surface 220 of the needle 211 so that barb-like appendages can be machined at other angles and directions. The angle of the chamfer 220 can range from less than 0 degrees when measured with respect to the axis of the needle 211 to greater than 180 degrees, where “less than” and “greater than” demonstrate an expanding internal or external taper.

In the side view and end view of FIGS. 22A-B, respectively, another embodiment for grasping tissue is shown that does not have unidirectional barb-like appendages. Here, near the distal end 221 of the needle a corset-type construction 222 is formed on a tube 223 perpendicular to the axis of the tube 223. Slots 228 (shown in FIG. 22B) are added so that grippers 230 are formed. Upon insertion of a smaller plunger-like device 224, the grippers 230 move away from the axis 226 (direction of movement shown in phantom) and thus into the tissue to grasp. The grippers 230 can be machined into many different shapes for different applications, such as a point-like one 232 shown in FIG. 22C. This embodiment demonstrates a method for accomplished this general idea of grasping tissue through expansion.

A further method of grasping tissue is depicted in the embodiment of FIG. 23. Utilizing micromachining and fabrication techniques, a plurality barb-like appendages 234 are formed to project from a surface 235. The size, shape, number, and density of these barbs 234 are a function of their ability to grasp specific kinds of tissue under specific circumstances. These barb-like appendages 234 can be machined not only on the surface 235 but also on a separate piece of material that would be attached to the location on the needle where gripping is desired. Moreover, these barb-like appendages can be formed on the Gecko feet 218 of the embodiment illustrated in FIG. 20.

In a related embodiment, these barb-like appendages are formed with a tissue-dependent design, that is, utilizing a specific design to optimize grasping of one type of tissue preferentially over another.

Movements other than rotation about the axis of the needle are also utilized to make the initial tissue grasp. Two of these other methods are shown in FIGS. 24A-B. In FIG. 24A the appendage 237 is rotated about an axis 238 perpendicular to the longitudinal axis 239 of the needle to arrive at a new position 236 (shown in phantom). In FIG. 24B, the appendage 237 is moved laterally, as shown by the arrow 242, in a direction perpendicular to the needle axis 239 to another position 240 (shown in phantom). These movements can be combined, also.

In FIG. 25 is shown a hook 244, similar to a fishhook, for grasping tissue. The hook 244, similar to a fishhook, emerges from a surface 245 at an oblique angle, as shown in FIG. 25. From the point of emergence 243, the hook 244 then changes direction to end up generally pointing in an opposite direction.

In an different embodiment, a device and method for determining when a certain tissue was passed through is illustrated and described. Here, the distal end face 212 of the needle 211 is shown passing through a first tissue layer 248. A compartment 252 is formed by the first tissue layer 248 and a second tissue layer 250. When that compartment 252 has a pressure difference, either a relative pressure differential, such as to the compartment that the needle was in previous to passing through the first tissue layer 248, or with respect to atmospheric pressure, then a transducer 246 placed in the end of the needle would be able to measure a change in pressure. The transduced pressure can be displayed to the user via a computer; however, that is the specific embodiment and other methods for feeding back information known to those skilled in the art can be used.

A further embodiment using the principles of pinching and grasping is shown in FIG. 27a. Here, an outer needle 258 rotates about its longitudinal axis 259 in a direction opposite the direction of rotation of an inner needle 260 (shown clockwise in figure as 262). Alternatively, the inner needle 260 can remain stationary or rotate in the same direction as the outer needle but at a different rotational rate. This actual or relative counter-rotating action will catch the tissue 206 between the two facing surfaces of the two needles (provided the geometry is appropriate to the tissue and situation) as seen in the enlarged view of FIG. 27B.

The counter-rotating embodiment of FIGS. 27A-B can be made more aggressive through the addition of barb-like appendages 270 and 272 (shown in FIG. 27C) that are configured to grab specific kinds of tissue 206.

To enable a user to keep cannula, tubes, catheters, grafts, stents, and other devices residing in a desired position or location, and to enable easy removal at the appropriate time, grasping mechanisms can be added to the outside wall 274 adjacent the distal end of the needle 211, as seen in FIG. 28A. In order to position this device into position, sheathing may be required, perhaps with another needle 276 as seen in FIG. 28B which then can be withdrawn so that the first needle can be moved such that the grasping mechanisms engage.

From the foregoing it will be appreciated that, although specific embodiments of the invention have been described herein for purposes of illustration, various modifications may be made without deviating from the spirit and scope of the invention. For example, the disclosed embodiments of the invention will find application outside the embodiments described above, such as a means of locating pain generators. Probes, such as electrodes, thermal transducers, guided discography proves, and fiber optics, to name a few, can be advanced through the cannula to view the epidural space and to localize and differentiate pain generator sites. Accordingly, the invention is not to be limited except as by the appended claims and the equivalents thereof.

What is claimed is:

1. A device for grasping tissue, comprising:
a tubular member having at a distal tip an annular surface surrounding a terminal port; and
at least one barb projecting at an angle from the annular surface of the tubular member, each at least one barb having a sharp edge configured to insert into the tissue and grasp the tissue as the tubular member is rotated about a longitudinal axis and to enable release of the tissue without puncturing the tissue.

2. The device of claim 1, wherein the at least one barb includes a plurality of barbs spaced around the annular surface.

3. The device of claim 2 wherein the plurality of barbs are unidirectional with respect to one another.

4. The device of claim 1 wherein the tubular member comprises a cannula.

5. The device of claim 1 wherein the annular surface is a blunt surface with the barbs projecting at an angle from the annular surface.

6. The device of claim 1, further comprising a peripheral ring defining a reduced diameter portion on an inner surface of the tubular member adjacent to the distal tip.

7. A device for grasping tissue, comprising:
a tubular member having at a distal tip an annular surface surrounding a terminal port;
a plurality of barbs each having a sharp edge projecting at an angle from the annular surface of the tubular member;
a first lateral port formed in an external wall surface of the tubular member adjacent to the annular surface; and
a second lateral port formed in an external wall surface of the tubular member and spaced away from the annular surface.

8. A device for grasping tissue, the device comprising:
a cannula having at a distal tip an annular surface surrounding a terminal port; and
a plurality of grasping members each projecting from the cannula and configured to grasp tissue and to release tissue without puncturing the tissue.

9. The device of claim 8 wherein the grasping members comprise barbs formed on the annular surface that are unidirectional.

10. The device of claim 9 wherein an angle at which the barbs project from the annular surface is an acute angle.

11. A spinal delivery system to deliver a tool through tissue, the system comprising:
a tube having a longitudinal axial bore and, at a distal tip, an annular surface surrounding a terminal port;
a housing secured to a proximal end of the tube, the housing having an internal cavity with an aperture formed in a proximal surface thereof opposite the proximal end of the tube;
a tool sized and shaped to be slidably received within the bore of the tube and having a blunt distal tip portion sized to pass through the terminal port in the distal tip of the tube and a proximal end portion sized to pass through the aperture in the proximal surface of the housing, the tool mounted in the housing to move between an extended position wherein the distal tip portion extends beyond the distal tip of the tube and a retracted position wherein the distal tip portion is withdrawn inside the tube; and
a resilient compression member mounted in the housing and configured to engage the tool when the tool is at an intermediate position between the extended position and the retracted position to thereby urge the tool into the extended position.

12. An epidural grasping device, comprising:
a cannula having at a distal tip an annular surface surrounding a terminal port;
a plurality of barbs each projecting a sharp edge at an angle from the annular surface of the cannula;
a first lateral port formed in an external wall surface of the cannula adjacent to the annular surface; and
a second lateral port formed in an external wall surface of the cannula and spaced away from the annular surface.

13. The epidural grasping device of claim 12 wherein the sharp edges of the barbs are structured to engage tissue presented at the annular surface of the cannula by rotation of the cannula about a longitudinal axis.

14. A spinal tool delivery system, comprising:
a cannula having at a distal tip an annular surface surrounding a terminal port;
a housing secured to a proximal end of the cannula, the housing having an internal cavity with an aperture formed in a proximal surface thereof opposite the proximal end of the cannula;
a stylet having a blunt distal tip portion sized to pass through the terminal port in the distal tip of the cannula and a proximal end portion sized to pass through the aperture formed in the proximal surface of the housing, the stylet mounted between the distal tip of the cannula and the proximal end of the housing and movable between first loaded position having the proximal end portion thereof projected a predetermined distance from the proximal surface of the housing, and second discharged position having the distal tip portion thereof projected a predetermined distance from the distal tip of the cannula; and
a resilient compression mechanism compressed between a laterally protruding surface of the stylet and the proximal surface of the housing when the proximal end portion of the stylet is projected in the first loaded position the predetermined distance from the proximal surface of the housing, whereby the resilient compression mechanism applies a predetermined pre-load force on the stylet to urge the stylet to project in the second discharged position the distal tip portion thereof the predetermined distance from the distal tip of the cannula.

15. A spinal needle system, comprising:
a cannula having a bore terminating at a distal tip in an inner peripheral ring of reduced diameter surrounding a terminal port, the inner peripheral ring forming an annular surface in the terminal port;
a plurality of sharp-edged barbs projecting at an angle from the annular surface of the cannula and circumferentially aligned relative to a longitudinal axis of the bore of the cannula;
a housing formed of a distal housing portion coupled to a proximal portion of the cannula and a proximal housing portion releaseably coupled to the distal housing portion, the distal and proximal housing portions enclosing an internal cavity with an aperture formed in a surface of the proximal housing portion opposite from the distal housing portion;
a stylet having a blunt distal tip portion sized to pass through the inner peripheral ring surrounding the terminal port at the distal tip of the cannula, a shoulder portion at a predetermined setback distance from the extent of the blunt distal tip, the shoulder portion sized to interfere with the inner peripheral ring, and at a proximal end an indicator portion sized to pass through the aperture formed in the proximal housing portion, the stylet mounted between the distal tip of the cannula and the proximal end of the proximal housing portion and movable between a first arrangement having the proximal end portion thereof projected from the proximal surface of the housing, and a second arrangement having the distal tip portion thereof projected from the distal tip of the cannula and the shoulder portion in contact with the inner peripheral ring; and
a resilient compression mechanism compressed between a laterally protruding rigid surface of the stylet and the surface of the housing having the aperture formed therein when the indicator portion at the proximal end of the stylet is projected from the proximal surface of the housing in the first arrangement, whereby the resilient compression mechanism applies a predetermined pre-load force on the laterally protruding rigid surface of the stylet to urge the stylet to project in the second arrangement the distal tip portion thereof from the distal tip of the cannula.

16. A method of using a cannula having at least one barb projecting from a distal surface thereof, the method comprising:
inserting the cannula through a first layer of tissue;
detecting contact of the distal surface of the cannula with a second layer of tissue; and
rotating the cannula in a first direction about a longitudinal axis to urge the at least one barb into engagement with the second layer of tissue.

17. The method of claim 16, further comprising:
initially slidably receiving within a bore of the cannula a tool sized and shaped to be slidably received within the bore of the cannula and having a distal tip portion sized and shaped to pass through an annular port in the distal surface of the cannula; and
after the at least one barb is engaged with the second layer of tissue, passing the distal tip portion of the tool through the annular port in the distal surface of the cannula.

18. The method of claim 17 wherein passing the distal tip portion of the tool through the annular port in the distal surface of the cannula includes expanding a compressed resilient compression member against a surface of the tool to urge the distal tip portion of the tool through the annular port.

19. The method of claim 18 wherein the detecting contact of the distal surface of the cannula with a second layer of tissue includes visually detecting when the distal surface of the cannula contacts the second layer of tissue.

20. The method of claim 18, further comprising before detecting contact of the distal surface of the cannula with a second layer of tissue, visually detecting when the distal surface of the cannula penetrates the first layer of tissue.

21. The method of claim 18, further comprising disengaging the at least one barb from engagement with the second layer of tissue by rotating the cannula about the longitudinal axis in a second direction opposite from the first direction.

22. A method of using a spinal needle delivery system comprising a cannula having at least one barb projecting from a distal surface thereof and a blunt stylet projecting from a portal in the distal surface thereof under pressure from a resilient biasing member, the blunt stylet movable relative to the distal surface of the cannula by compression and expansion of the resilient biasing member; an indicator portion generating an indication as a function of a degree of projection of the blunt stylet relative to the portal in the distal surface of the cannula; and a cannula lock coupling an adhesive band to the cannula, the method comprising:
in a previously perforated first layer of relatively high resistance tissue, enlarging the perforation sufficiently to permit entry of a distal tip of the blunt stylet;
stabilizing the spinal needle delivery system relative to the enlarged perforation;
advancing the distal tips of the blunt stylet and the cannula into and through the enlarged perforation in the layer of relatively high resistance tissue;
using the indicator, determining that the distal tip of the blunt stylet has passed through the enlarged perforation in the layer of relatively high resistance tissue into a space of relatively low resistance;
securing the cannula lock to the shaft of the cannula, thereby fixing the adhesive band relative to the cannula and advancing the distal tips of the blunt stylet and the cannula through the space of relatively low resistance and into contact with a second relatively high resistance tissue;
using the indicator, determining that the distal tip of the cannula has contacted the second relatively high resistance tissue;
rotating the cannula into an engaged position by rotating the cannula in a direction to engage the barbs with the second relatively high resistance tissue until resistance to continued rotation is encountered;
supporting the cannula in the engaged position while advancing the cannula lock and adhesive band along the shaft of the cannula until the adhesive band contacts but does not depress the first layer of relatively high resistance tissue adjacent to the enlarged perforation;
adhering the adhesive band to the first layer of relatively high resistance tissue; and
supporting the cannula of the spinal needle delivery system.

23. The method of claim 22, further comprising retrieval of the spinal needle delivery system by:
separating the adhesive band from the first layer of relatively high resistance tissue;
rotating the cannula into a disengaged position by rotating the spinal needle delivery system in a direction to disengage the barbs from the second relatively high resistance tissue; and
withdrawing the spinal needle delivery system from the perforation.

24. A spinal delivery system to deliver a tool into tissue, the system comprising:
a tube having a longitudinal axial bore and, at a distal tip, an annular surface surrounding a terminal port, and member for grasping and releasing tissue without puncturing the tissue;
a housing secured to a proximal end of the tube, the housing having an internal cavity with an aperture formed in a proximal surface thereof opposite the proximal end of the tube;
a tool sized and shaped to be slidably received within the bore of the tube and having a blunt distal tip portion sized to pass through the terminal port in the distal tip of the tube and a proximal end portion sized to pass through the aperture in the proximal surface of the housing, the tool mounted in the housing to move between an extended position wherein the distal tip portion extends beyond the distal tip of the tube and a retracted position wherein the distal tip portion is withdrawn inside the tube; and
a resilient compression member mounted in the housing and configured to engage the tool when the tool is at an intermediate position between the extended position and the retracted position to thereby urge the tool into the extended position.

25. A surgical device, comprising:
a cannula having at a distal tip a terminal port and means for grasping and releasing tissue without puncturing through the tissue.

26. The device of claim 25, wherein the grasping means comprises adhesive material at the distal tip.

27. The device of claim 26, wherein the adhesive material is selectively activated and deactiveated to grasp and release tissue respectively by one of either chemical message and electric current.

28. The device of claim 25, wherein the grasping means comprises means configured for applying vacuum to tissue.

29. The device of claim 28, further comprising a skirt formed at the distal tip.

30. The device of claim 25, wherein the grasping means comprises material configured to utilize van der Waals forces.

31. The device of claim 25, wherein the grasping means comprises arms mounted at the distal tip to swing towards each other and means on at least one arm to grasp tissue.

32. The device of claim 25, wherein the grasping means comprises arms mounted at the distal tip to move laterally towards each other and means on at least one arm to grasp tissue.

33. The device of claim 25, wherein the grasping means comprises grasping members concentrically mounted to rotate about a rotational axis and cooperate to grasp tissue.

34. The device of claim 33, wherein the grasping members are counter-rotated.

35. The device of claim 33 wherein the grasping members are rotated at different speeds in the same rotational direction.

36. A surgical device, comprising:
a cannula having at a distal tip a terminal port and means for grasping and releasing tissue; and
means for providing feedback to a user on the location of the distal tip with respect to the tissue.

37. The device of claim 36, wherein the feedback means comprises means for measuring a pressure differential at the distal tip.

38. The device of claim 36, wherein the pressure differential measuring means comprises a pressure transducer.

39. A device for grasping and releasing tissue, comprising:
a tube having a longitudinal axial bore and a distal end piece configured to have radially outward facing barbs extending from a necked-down portion of the distal end piece, the necked-down portion configured to expand outward; and
a plunger slidably mounted in the longitudinal axial bore of the tube and configured to slide into the necked-down portion of the distal end piece and urge the barbs to extend outward.

40. The device of claim 39, wherein the distal end piece is formed of resilient material to urge the barbs to retract inward.
